(19) United States
(12) Patent Application Publication
(10) Pub. No.: US 2022/0396543 A1
(43) Pub. Date: Dec. 15, 2022

(54) Method for Separating Water-Containing Crystals, Method for Producing Methacrylic Acid, and Method for Producing Methacrylic Acid Ester

(71) Applicant: Mitsubishi Chemical Corporation, Tokyo (JP)

(72) Inventors: Ryutaro Ishibashi, Tokyo (JP); Koji Kanaya, Tokyo (JP); Yugo Mizokoshi, Tokyo (JP)

(73) Assignee: Mitsubishi Chemical Corporation, Tokyo (JP)

(21) Appl. No.: 17/891,241

(22) Filed: Aug. 19, 2022

Related U.S. Application Data
(63) Continuation of application No. PCT/JP2021/007176, filed on Feb. 25, 2021.

(30) Foreign Application Priority Data
Feb. 26, 2020 (JP) 2020-030811

(51) Int. Cl. C07C 51/43

(52) U.S. Cl. CPC C07C 51/43; C07B 2200/13

(57) ABSTRACT

Provided is a method for separating water-containing crystals, the method including: a step of generating water-containing crystals from a mixed liquid including water, acetic acid, and methacrylic acid; and a step of separating the crystals, in which the proportion by mass of methacrylic acid with respect to the total mass of water, acetic acid, and methacrylic acid in the mixed liquid is 0.09% by mass or more and less than 0.60% by mass.

### TECHNICAL FIELD

The present invention relates to a method for separating water-containing crystals, the method including a step of separating water-containing crystals from a mixed liquid including acetic acid, water, and methacrylic acid; a method for producing methacrylic acid, the method being included in the step of separating water-containing crystals by the above-described separation method; and a method for producing a methacrylic acid ester, the method including a step of producing methacrylic acid by the above-described method for producing methacrylic acid, and a step of producing a methacrylic acid ester from the methacrylic acid and methanol.

This application is a continuation application of International Application No. PCT/JP2021/007176, filed on Feb. 25, 2021, which claims the benefit of priority of the prior Japanese Patent Application No. 2020-030811, filed Feb. 26, 2020, the entire content of which is incorporated herein by reference.

### BACKGROUND ART

In a plant that industrially produces carboxylic acids and the like, large quantities of unreacted raw materials and waste liquids derived from carboxylic acid production processes and purification processes are generated. Such waste liquids include, as representative compounds, water, acetic acid, unreacted raw materials, and compounds by-produced in the above-described production processes. When treating such a waste liquid, since many organic compounds are included, it is necessary that the organic compounds are removed from the waste liquid or subjected to a detoxification treatment, and then only those compounds having no risk of polluting the environment, such as nitrogen, water, and carbon dioxide, are finally discharged.

As a method for treating such a waste liquid, for example, a method of combusting the organic compounds included in the waste liquid and discharging the resultant as water or carbon dioxide is known. However, the waste liquid may include a large amount of water, and when the waste liquid is subjected to a combustion treatment as it is, the treatment cost increases high. Therefore, it is preferable to separate water from the waste liquid in advance and lower the water concentration in the waste liquid. Regarding a method for separating water from a waste liquid as such, for example, a method of cooling industrial wastewater including acetic acid and water, depositing crystals, separating the crystals from the liquid, and thereby separating acetic acid and water, is described in Patent Document 1.

### CITATION LIST

### Patent Document

### Patent Document 1

Korean Patent No. 10-0725588

### SUMMARY OF INVENTION

### Technical Problem

There is a possibility that only water can be separated from industrial wastewater including acetic acid and water, by the method of Patent Document 1. However, according to an investigation made by the inventors of the present invention, it has been found that in the case of the method described in Patent Document 1, a certain amount or more of acetic acid may be included in the separated water. As such, when acetic acid is included in the water after separation, a complicated treatment for removing acetic acid from the water may be required again at the time of treating the water as wastewater. Thus, it is an object of the present invention is to provide a method for separating water-containing crystals, by which high-purity water can be separated from a mixed liquid including methacrylic acid, acetic acid, and water by a simpler method.

### Solution to Problem

In view of the above-described circumstances, the present inventors found that the above-described problems can be solved by carrying out an operation of generating water-containing crystals in a mixed liquid of acetic acid and water in a state of including a specific amount of methacrylic acid, and then separating the crystals, thus achieving the invention. That is, the gist of the present invention is as follows.

[1]

A method for separating water-containing crystals, the method including:

a step of generating water-containing crystals from a mixed liquid including water, acetic acid, and methacrylic acid; and

a step of separating the crystals,

in which a proportion by mass of methacrylic acid with respect to a total mass of water, acetic acid, and methacrylic acid in the mixed liquid is 0.09% by mass or more and less than 0.60% by mass.

[2]

The method for separating water-containing crystals according to [1],

in which a proportion by mass of water with respect to the total mass of water, acetic acid, and methacrylic acid in the mixed liquid is more than 79.94% by mass and 99.86% by mass or less.

[³]

The method for separating water-containing crystals according to [1] or [2],

in which a proportion by mass of acetic acid with respect to the total mass of water, acetic acid, and methacrylic acid in the mixed liquid is 0.05% by mass or more and 20.00% by mass or less.

[4]

The method for separating water-containing crystals according to any one of [1] to [3],

in which the step of generating the crystals is a step of generating water-containing crystals by cooling the mixed liquid.

[⁵]

The method for separating water-containing crystals according to [4],

in which a cooling temperature for the mixed liquid is −15° C. or higher and 10° C. or lower.

[6]

A method for producing methacrylic acid, the method including:

a step of separating water by the method according to any one of [1] to [5].

[7]

A method for producing a methacrylic acid ester, the method including:

a step of producing methacrylic acid by the method for producing methacrylic acid according to [6]; and

a step of producing a methacrylic acid ester from the methacrylic acid and methanol.

The present invention also includes the following aspects.

[1]

A method for separating water-containing crystals, the method including:

a step of generating water-containing crystals from a mixed liquid including water, acetic acid, and methacrylic acid; and

a step of separating the crystals.

[2]

The method for separating water-containing crystals according to [1],

in which a proportion by mass of methacrylic acid with respect to a total mass of water, acetic acid, and methacrylic acid in the mixed liquid is preferably 0.09% by mass or more and less than 0.60% by mass, more preferably 0.10% by mass or more and 0.55% by mass or less, even more preferably 0.12% by mass or more and 0.50% by mass or less, and particularly preferably 0.15% by mass or more and 0.40% by mass or less.

[3]

The method for separating water-containing crystals according to [1] or [2],

in which a proportion by mass of water with respect to the total mass of water, acetic acid, and methacrylic acid in the mixed liquid is preferably more than 79.94% by mass and 99.86% by mass or less, more preferably 85.00% by mass or more and 99.00% by mass or less, and particularly preferably 88.00% by mass or more and 95.00% by mass or less.

[4]

The method for separating water-containing crystals according to any one of [1] to [3],

in which a proportion by mass of acetic acid with respect to the total mass of water, acetic acid, and methacrylic acid in the mixed liquid is preferably 0.05% by mass or more and 20.00% by mass or less, more preferably 1.00% by mass or more and 15.00% by mass or less, and particularly preferably 5.00% by mass or more and 12.00% by mass or less.

[5]

The method for separating water-containing crystals according to any one of [1] to [4],

in which the step of generating the crystals is a step of generating water-containing crystals by cooling the mixed liquid.

[6]

The method for separating water-containing crystals according to [5],

in which a cooling temperature for the mixed liquid is preferably −15° C. or higher and 10° C. or lower, more preferably −10° C. or higher and 5° C. or lower, and particularly preferably −5° C. or higher and 0° C. or lower.

[7]

The method for separating water-containing crystals according to [5] or [6],

in which a temperature of the mixed liquid before cooling the mixed liquid is preferably −5° C. or higher and 15° C. or lower, more preferably 0° C. or higher and 10° C. or lower, and particularly preferably 2° C. or higher and 5° C. or lower.

[8]

The method for separating water-containing crystals according to any one of [5] to [7],

in which a cooling rate for the mixed liquid is preferably 0.01 K/min or greater and 2.00 K/min or less, more preferably 0.02 K/min or greater and 1.00 K/min or less, and particularly preferably 0.04 K/min or greater and 0.50 K/min or less.

[9]

The method for separating water-containing crystals according to any one of [5] to [8],

in which a time for maintaining the cooling temperature for the mixed liquid is preferably 20 minutes or more and 5 hours or less, more preferably 40 minutes or more and 4 hours or less, and particularly preferably 60 minutes or more and 3 hours or less.

[10]

The method for separating water-containing crystals according to any one of [1] to [9],

in which the proportion by mass of water with respect to a mass of the crystals is preferably 80% by mass or more and 100% by mass or less, more preferably 90% by mass or more and 100% by mass or less, and even more preferably 95% by mass or more and 100% by mass or less.

[11]

The method for separating water-containing crystals according to any one of [1] to [10],

in which in the step of separating the crystals, the crystals are separated by filtering a slurry containing the crystals and a mother liquor including acetic acid and methacrylic acid.

[12]

The method for separating water-containing crystals according to [11],

in which the mother liquor includes water, and

the proportion by mass of water with respect to a mass of the mother liquor is preferably more than 0% by mass and 80% by mass or less, and more preferably more than 0% by mass and 70% by mass or less.

[13]

The method for separating water-containing crystals according to any one of [1] to [12],

in which the crystals contain acetic acid, and

the proportion by mass of acetic acid with respect to the mass of the crystals is preferably more than 0 ppm and 40000 ppm or less, and more preferably more than 0 ppm and 30000 ppm or less.

[14]

The method for separating water-containing crystals according to any one of [1] to [13],

in which the crystals contain methacrylic acid, and

the proportion by mass of methacrylic acid with respect to the mass of the crystal is preferably more than 0 ppm and 800 ppm or less, and more preferably more than 0 ppm and 700 ppm or less.

[15]

A method for producing or purifying methacrylic acid, the method including:

a step of separating water-containing crystals by the method according to any one of [1] to [14].

[16]

The method for producing or purifying methacrylic acid according to [15], including a step of recovering methacrylic acid from a mother liquor including acetic acid and methacrylic acid after the step of separating water-containing crystals.

[17]

The method for producing or purifying methacrylic acid according to [15] or [16],

in which the methacrylic acid recovered by the step of recovering methacrylic acid includes acetic acid, and

the proportion by mass of acetic acid with respect to a mass of the recovered methacrylic acid is preferably more than 0 ppm and 100000 ppm or less, and more preferably more than 0 ppm and 50000 ppm or less.

[18]

The method for producing or purifying methacrylic acid according to any one of [15] to [17],

in which the methacrylic acid recovered by the step of recovering methacrylic acid includes water, and

the proportion by mass of water with respect to the mass of the recovered methacrylic acid is preferably more than 0 ppm and 10000 ppm or less, and more preferably more than 0 ppm and 5000 ppm or less.

[19]

A method for producing a methacrylic acid ester, the method including:

a step of producing or purifying methacrylic acid by the method for producing or purifying methacrylic acid according to any one of [15] to [18], and

a step of producing a methacrylic acid ester from methacrylic acid and methanol.

[20]

The method for producing a methacrylic acid ester according to [19],

in which the step of producing the methacrylic acid ester is carried out by using a fixed-bed reactor packed with an ion exchange resin.

[21]

The method for producing a methacrylic acid ester according to [19] or [20],

in which the amount of liquid passage of a raw material including methacrylic acid and methanol in the fixed-bed reactor is preferably 0.10 times or more and 10.0 times or less, and more preferably 0.20 times or more and 5.0 times or less, in terms of mass ratio with respect to an amount of the ion exchange resin.

### Advantageous Effects of Invention

According to the present invention, a method for separating water-containing crystals, by which high-purity water can be separated from a mixed liquid including methacrylic acid, acetic acid, and water by a simpler method, and a method for producing methacrylic acid using this method for separating water-containing crystals, and a method for producing a methacrylic acid ester using the method for producing methacrylic acid, can be provided.

### DESCRIPTION OF EMBODIMENTS

Hereinafter, embodiments of the present invention will be described in detail; however, the following description is an example of the embodiments of the present invention, and the present invention is not intended to be limited to these contents and can be carried out as various modifications to the extent of the gist of the invention.

In the method for separating water-containing crystals according to the present embodiment, a slurry including water-containing crystals and a liquid that is not crystallized (hereinafter, may be referred to as mother liquor) is obtained by generating crystals in a mixed liquid in a state in which methacrylic acid is present in the mixed liquid, and the crystals in the slurry are separated by separating the mother liquor from the slurry. In the present invention, the operation of producing a slurry including crystals and a mother liquor by generating crystals from a mixed liquid is referred to as a crystallization operation.

### Crystallization Operation

Crystals are deposited by subjecting a mixed liquid including water, acetic acid, and methacrylic acid to a crystallization operation. More specifically, crystals containing water as a main component are formed by generating crystals in a mixed liquid including water, acetic acid, and methacrylic acid. Incidentally, according to the present invention, it is meant by the crystals containing water as a main component that the proportion of water with respect to the total mass of the crystals is 80% by mass or more. In this way, a slurry including crystals and a mother liquor is produced.

The proportion by mass of water with respect to the total mass of water, acetic acid, and methacrylic acid in the mixed liquid before the crystallization operation is not particularly limited; however, in order to enable cooling with a general-purpose refrigerant that can be used relatively inexpensively, the proportion by mass of water is preferably more than 79.94% by mass, more preferably 85.00% by mass or more, and particularly preferably 88.00% by mass or more. When the concentration of water in the mixed liquid is higher, there is a tendency that high-purity water can be separated; however, in the case of the present invention, even when the concentration of water in the mixed liquid is low, high-purity water can be separated from the mixed liquid despite the fact that a certain extent of acetic acid is included in the mixed liquid. From this, from the viewpoint of suppressing the operating cost for the crystallization operation, the proportion by mass of water with respect to the total mass of water, acetic acid, and methacrylic acid in the mixed liquid is preferably 99.86% by mass or less, more preferably 99.00% by mass or less, and particularly preferably 95.00% by mass or less. More specifically, the proportion by mass of water with respect to the total mass of water, acetic acid, and methacrylic acid in the mixed liquid is preferably more than 79.94% by mass and 99.86% by mass or less, more preferably 85.00% by mass or more and 99.00% by mass or less, and particularly preferably 88.00% by mass or more and 95.00% by mass or less.

The proportion by mass of acetic acid with respect to the total mass of water, acetic acid, and methacrylic acid in the mixed liquid before the crystallization operation is not particularly limited; however, in the case of the present invention, since high-purity water can be separated from the mixed liquid, the proportion by mass of acetic acid is preferably 0.05% by mass or more, more preferably 1.00% by mass or more, and even more preferably 5.00% by mass or more. On the other hand, in order to allow cooling with a general-purpose refrigerant that can be used relatively inexpensively, the proportion by mass of acetic acid with respect to the total mass of water, acetic acid, and methacrylic acid in the mixed liquid is preferably 20.00% by mass or less, more preferably 15.00% by mass or less, and particularly preferably 12.00% by mass or less. More specifically, the proportion by mass of acetic acid with respect to the total mass of water, acetic acid, and methacrylic acid in the mixed liquid is preferably 0.05% by mass or more and 20.00% by mass or less, more preferably 1.00% by mass or more and 15.00% by mass or less, and particularly preferably 5.00% by mass or more and 12.00% by mass or less.

The proportion by mass of methacrylic acid with respect to the total mass of water, acetic acid, and methacrylic acid in the mixed liquid before the crystallization operation is 0.09% by mass or more and less than 0.60% by mass. By setting the proportion by mass of methacrylic acid to be in the above-described range, it is possible to separate high-purity water. This mechanism is not clearly understood; however, the following reasons may be considered.

The purity of the crystals obtained by the crystallization operation is determined by the solid-liquid separability based on the difference in crystal shape, and the amount of impurities incorporated into the crystals. According to the present invention, it is speculated that the presence of a trace amount of methacrylic acid in the mixed liquid favorably works on the crystal shape and the amount of internal impurities obtainable during the crystallization operation, and the content of impurities in the obtained crystals is lowered. Incidentally, a specific amount of methacrylic acid is included in the mixed liquid to be subjected to the crystallization operation for the above-mentioned reasons, and methacrylic acid may also be included in the water after separation from the mixed liquid. However, since methacrylic acid is easily biodegraded, even when methacrylic acid is included in the water after separation, methacrylic acid can be easily removed. Furthermore, the total amount of acetic acid and methacrylic acid contained in the water after separation according to the present embodiment tends to be lowered as compared with the concentration of acetic acid after separation when water is separated from the mixed liquid including acetic acid and water without using methacrylic acid. Therefore, by using methacrylic acid, crystals of water having higher purity can be obtained as compared with the case where methacrylic acid is not used.

Above all, the proportion by mass of methacrylic acid with respect to the total mass of water, acetic acid, and methacrylic acid in the mixed liquid before the crystallization operation is preferably 0.09% by mass or more, more preferably 0.10% by mass or more, even more preferably 0.12% by mass or more, and particularly preferably 0.15% by mass or more. On the other hand, the proportion by mass of methacrylic acid with respect to the total mass of water, acetic acid, and methacrylic acid in the mixed liquid is preferably less than 0.60% by mass, more preferably 0.55% by mass or less, even more preferably 0.50% by mass or less, still more preferably 0.45% by mass or less, particularly preferably 0.40% by mass or less, and most preferably 0.30% by mass or less. More specifically, the proportion by mass of methacrylic acid with respect to the total mass of water, acetic acid, and methacrylic acid in the mixed liquid is preferably 0.09% by mass or more and less than 0.60% by mass, more preferably 0.10% by mass or more and 0.55% by mass or less, even more preferably 0.12% by mass or more and 0.50% by mass or less, and particularly preferably 0.15% by mass or more and 0.40% by mass or less.

Incidentally, when a waste liquid including methacrylic acid occurs as a result of treating industrial wastewater by the method for separating water-containing crystals of the present invention, it is preferable to produce the mixed liquid by mixing a waste liquid including water and acetic acid with the waste liquid including methacrylic acid and adjusting the methacrylic acid concentration. As a result, the methacrylic acid used in the method for separating water-containing crystals of the present invention can be reutilized.

The mixed liquid before the crystallization operation may also contain other components in addition to water, acetic acid, and methacrylic acid. Examples of the other components include acrylic acid, propionic acid, formic acid, maleic anhydride, methanol, and methacrolein.

The total concentration of the other components included in the mixed liquid before the crystallization operation is not particularly limited; however, in order to separate high-purity water from the mixed liquid, the total concentration is preferably 2.00% by mass or less, more preferably 1.00% by mass or less, and particularly preferably 0.50% by mass or less, with respect to the total mass of the mixed liquid. On the other hand, the total concentration of the other components is not particularly limited; however, in order to separate high-purity water from the mixed liquid, the total concentration is preferably 0% by mass or more, more preferably more than 0% by mass, and particularly preferably 0.01% by mass or more, with respect to the total mass of the mixed liquid.

The preferred ranges of the water concentration, acetic acid concentration, methacrylic acid concentration, and the concentration of the other components in the mixed liquid are as mentioned above; however, it is preferable to adjust the concentration of each of the components to be within these preferred ranges so that the total amount is 100% by mass with respect to the total mass of the mixed liquid.

The apparatus for performing crystallization is not particularly limited as long as it can generate crystals in the mixed liquid and deposit water-containing crystals, and any known apparatus can be used. For example, known devices described in “Chemical Engineering Handbook, Revised 5th Edition (1988)” can be used. Above all, it is preferable to use a jacket type crystallization tank. In the jacket type crystallization tank, the inside of the crystallization tank is cooled by the flow of the refrigerant in the jacket, and as a result, crystals are deposited on the wall surface of the crystallization tank. The type of the crystallization operation may be either batch type or continuous type. Hereinafter, the form when a crystallization tank is used will be described; however, the following conditions at the time of crystallization are also effective when other crystallization apparatuses are used.

The crystallization operation can also be performed by cooling the mixed liquid. In this case, the crystallization temperature, that is, the cooling temperature is not particularly limited; however, the crystallization temperature is preferably −15° C. or higher, more preferably −10° C. or higher, and particularly preferably −5° C. or higher, in order to reduce fluctuations in the operating conditions due to air temperature. On the other hand, in order to reduce the fluctuations in the operating conditions even when the air temperature drops, the crystallization temperature is preferably 10° C. or lower, more preferably 5° C. or lower, and particularly preferably 0° C. or lower. Incidentally, the crystallization temperature means the temperature of the mixed liquid during crystallization. More specifically, the cooling temperature for the mixed liquid is preferably −15° C. or higher and 10° C. or lower, more preferably −10° C. or higher and 5° C. or lower, and particularly preferably −5° C. or higher and 0° C. or lower.

The temperature of the mixed liquid when the mixed liquid is supplied to the crystallization tank (that is, the temperature of the mixed liquid before cooling the mixed liquid) is not particularly limited; however, in order to prevent the formation of crystals in the mixed liquid before being supplied to the crystallization tank, the temperature is preferably −5° C. or higher, more preferably 0° C. or higher, and particularly preferably 2° C. or higher. On the other hand, in order not to affect the liquid temperature in the crystallization tank, the temperature of the mixed liquid before cooling the mixed liquid is preferably 15° C. or lower, more preferably 10° C. or lower, and particularly preferably 5° C. or lower. More specifically, the temperature of the mixed liquid before cooling the mixed liquid is preferably −5° C. or higher and 15° C. or lower, more preferably 0° C. or higher and 10° C. or lower, and particularly preferably 2° C. or higher and 5° C. or lower.

When the crystallization operation is performed by cooling the mixed liquid, the cooling rate for the mixed liquid is not particularly limited; however, in order to obtain crystals in a short residence time, the cooling rate is preferably 0.01 K/min or greater, more preferably 0.02 K/min or greater, and particularly preferably 0.04 K/min or greater. On the other hand, in order to further reduce the impurity concentration, the cooling rate for the mixed liquid is preferably 2.00 K/min or less, more preferably 1.00 K/min or less, and particularly preferably 0.50 K/min or less. More specifically, the cooling rate for the mixed liquid is preferably 0.01 K/min or greater and 2.00 K/min or less, more preferably 0.02 K/min or greater and 1.00 K/min or less, and particularly preferably 0.04 K/min or greater and 0.50 K/min or less.

When the crystallization operation is performed by cooling the mixed liquid, the time for maintaining the cooling temperature at the time of crystallization is not particularly limited as long as crystals containing water as a main component are deposited; however, in order to obtain a sufficient amount of crystals, the time is preferably 20 minutes or more, more preferably 40 minutes or more, and particularly preferably 60 minutes or more. On the other hand, in order to obtain crystals in a short time, the time is preferably 5 hours or less, more preferably 4 hours or less, and particularly preferably 3 hours or less. More specifically, the time for maintaining the cooling temperature for the mixed liquid is preferably 20 minutes or more and 5 hours or less, more preferably 40 minutes or more and 4 hours or less, and particularly preferably 60 minutes or more and 3 hours or less. When the crystallization operation is performed in the manner of continuous type, the time for maintaining the cooling temperature means the retention time of the crystallization tank.

The jacket type crystallization tank may be provided with a scraping type stirring blade for scraping the crystals deposited on the wall surface of the crystallization tank. As described above, the crystals are mainly deposited on the wall surface of the crystallization tank; however, as the amount of crystals deposited on the wall surface increases, it is difficult for new crystals to be deposited. Therefore, the crystals can be efficiently deposited by rotating the scraping type stirring blade at the time of the crystallization operation and scraping the crystals deposited on the wall surface of the crystallization tank.

### Separation of Crystals and Mother Liquor

By the crystallization operation, a slurry including crystals containing water as a main component and a mother liquor is obtained, and by filtering the slurry, the crystals can be obtained by separating the mother liquor. That is, since the main component of the crystals is water, as a result, water can be separated from the mixed liquid including methacrylic acid, water, and acetic acid.

The proportion by mass of water with respect to the mass of the water-containing crystals is preferably 80% by mass or more and 100% by mass or less, more preferably 90% by mass or more and 100% by mass or less, and even more preferably 95% by mass or more and 100% by mass or less. When the content of water in the crystal is in the above-described range, a treatment for removing acetic acid from the water is unnecessary, and therefore, the labor and cost required for a wastewater treatment can be easily reduced.

When water is included in the mother liquor, the proportion by mass of water with respect to the mass of the mother liquor is preferably more than 0% by mass and 80% by mass or less, and more preferably more than 0% by mass and 70% by mass or less. When the content of water in the mother liquor is in the above-described range, a complicated treatment for removing acetic acid from the water can be reduced, and the cost for combustion treatment of wastewater including acetic acid can be suppressed.

The water-containing crystals may have acetic acid or methacrylic acid attached thereto.

When acetic acid is attached to the water-containing crystals, the proportion by mass of acetic acid with respect to the mass of the crystals is preferably more than 0 ppm and 40000 ppm or less, and more preferably more than 0 ppm and 30000 ppm or less.

When methacrylic acid is attached to the water-containing crystals, the proportion by mass of methacrylic acid with respect to the mass of the crystals is preferably more than 0 ppm and 800 ppm or less, and more preferably more than 0 ppm and 700 ppm or less.

The method for separating the crystals and the mother liquor is not particularly limited as long as it is a method capable of separating the crystals and the mother liquor, and for example, known methods such as a filtration method and a centrifugal separation method can be utilized. Examples of the apparatus for performing separation include KCP apparatuses listed in Chuzo Shimizu: “Purification of Organic Compounds by KUREHA Continuous Crystal Purification Apparatus”, Chemical Engineering, Vol. 27, No. 3 (1982), p. 49. The type of the separation operation may be either batch type or continuous type. Furthermore, the separation of the crystals and the mother liquor in the slurry may be carried out in a state in which the slurry is stopped, or may be carried out while moving the slurry.

As such, according to the method for separating water-containing crystals of the present invention, high-purity water can be separated from a mixed liquid including methacrylic acid, water, and acetic acid. Therefore, for example, when the water after separation is disposed of, it is considered possible to dispose of the obtained water without applying a complicated treatment. On the other hand, since the amount of water in the mother liquor has been significantly reduced, the complicated treatment of removing acetic acid from the water can be reduced, and the cost for a combustion treatment of wastewater including acetic acid can be suppressed.

### Method for Producing Methacrylic Acid

A method for producing methacrylic acid of the present invention includes a step of separating water by the method for separating water-containing crystals of the present invention. According to the method for producing methacrylic acid of the present invention, as the cost for a wastewater treatment is lowered, methyl methacrylate can be produced by an inexpensive method with less impact on the environment.

It is preferable that the method for producing methacrylic acid of the present invention includes a step of recovering methacrylic acid from a mother liquor including acetic acid and methacrylic acid, after separating water. As a result, the methacrylic acid used in the method for separating water-containing crystals of the present invention can be recovered.

It is preferable that the method for producing methacrylic acid of the present invention includes a step of recovering methacrylic acid from a mother liquor including acetic acid and methacrylic acid, after separating water. As a result, the methacrylic acid used in the method for separating water-containing crystals of the present invention can be recovered.

The methacrylic acid recovered in the step of recovering may include acetic acid and water.

When the recovered methacrylic acid includes acetic acid, the proportion by mass of acetic acid with respect to the mass of the recovered methacrylic acid is preferably more than 0 ppm and 100000 ppm or less, and more preferably more than 0 ppm and 50000 ppm or less.

When the recovered methacrylic acid includes water, the proportion by mass of water with respect to the mass of the recovered methacrylic acid is preferably more than 0 ppm and 10000 ppm or less, and more preferably more than 0 ppm and 5000 ppm or less.

### Method for Producing Methyl Methacrylate

A method for producing methyl methacrylate of the present invention may include the method for producing methacrylic acid of the present invention. Specifically, the method includes a step of producing methacrylic acid, a step of producing methyl methacrylate from methacrylic acid and methanol, and a step of separating methyl methacrylate from the mixed liquid. According to the above-described method, as the cost for a wastewater treatment is lowered, methyl methacrylate can be produced by an inexpensive method with less impact on the environment.

For example, the methacrylic acid used in the method for separating water-containing crystals of the present invention is recovered by extraction and distillation operations and the like, and the methacrylic acid is subjected to an esterification reaction with methanol in the presence of an acid catalyst to obtain methyl methacrylate. In the esterification reaction, it is preferable to use a catalyst. The catalyst to be used is preferably an acid catalyst, and above all, sulfuric acid or an ion exchange resin can be used. As the ion exchange resin, a strongly acidic cation exchange resin is preferable. Specific examples of the strongly acidic cation exchange resin include DIAION (registered trademark), PK216, RCP12H (manufactured by Mitsubishi Chemical Corporation), LEWATIT (registered trademark), K2431 (manufactured by Bayer AG), AMBERLYST (registered trademark), and 15WET (manufactured by Rohm and Haas Japan K. K.). These may be used singly, or two or more kinds thereof may be used in combination.

When the esterification reaction is carried out using a fixed-bed reactor packed with an ion exchange resin, the flow direction of the reaction fluid in the esterification reaction may be either vertically upward or vertically downward and can be appropriately selected. When the swelling of the ion exchange resin used as the acid catalyst of the esterification reaction is large, the flow direction of the reaction fluid is preferably vertically upward. When the reaction fluid forms a non-uniform phase, the flow direction of the reaction fluid is preferably vertically downward.

When the esterification reaction is carried out using a fixed-bed reactor packed with an ion exchange resin, the amount of liquid passage of the raw material including methacrylic acid and methanol is preferably 0.10 times or more, and more preferably 0.20 times or more, in terms of mass ratio with respect to the amount of the ion exchange resin. Furthermore, the amount of liquid passage of the raw material is preferably 10.0 times or less, and more preferably 5.0 times or less, in terms of mass ratio with respect to the amount of the ion exchange resin. More specifically, the amount of liquid passage of the raw material including methacrylic acid and methanol is preferably 0.10 times or more and 10.0 times or less, and more preferably 0.20 times or more and 5.0 times or less, in terms of mass ratio with respect to the amount of the ion exchange resin.

### EXAMPLES

Hereinafter, the present invention will be described in detail based on Examples; however, the present invention is not intended to be limited by these.

In the following Examples and Comparative Examples, a jacket type crystallization tank having a volume of 1 L and equipped with a scraping type stirring blade was used.

### Example 1

800 g of a mixed liquid having an acetic acid concentration of 8.0% by mass, a methacrylic acid concentration of 0.2% by mass, and a water concentration of 91.8% by mass with respect to the total mass of the mixed liquid, was introduced into the crystallization tank. The rotation speed of the scraping type stirring blade was set to 200 rpm, and the refrigerant flowing through the jacket part of the crystallization tank was cooled at a cooling rate of 0.067 K/min. At the stage where the temperature of the mixed liquid in the crystallization tank reached −5° C., the temperature was maintained for 2 hours, and crystals were deposited. Subsequently, about 250 ml of the obtained slurry liquid consisting of crystals and a mother liquor was transferred into a glass tube with a filter (manufactured by Sibata Scientific Technology, Ltd., model number: GO-51, filter pore size 40 μm) by using a vacuum pump, and filter filtration was performed in an incubator maintained at a temperature of 20° C. for 30 minutes.

The concentration of impurities in the crystals remaining on the filter part was measured by gas chromatography (GC). The obtained results are shown in Table 2. The measurement by gas chromatography was carried out under the conditions shown in Table 1. Furthermore, the column temperature was maintained at 40° C. for 5 minutes, subsequently raised at a rate of 10° C./min, maintained for 15 minutes at the time point when the temperature reached 100° C., subsequently raised again at a rate of 10° C./min, and maintained for 5 minutes at the time point when the temperature reached 220° C.

TABLE 1

| Apparatus | Model number: 7890B manufactured |
|---|---|
| | by Agilent Technologies, Inc. |
| Column | DB-FFAP (length 30 m, |
| | inner diameter 0.32 mm, |
| | membrane thickness 0.25 mm) |
| Column inlet pressure | 52.48 kPa |
| Column flow rate | 84.3 ml/m |
| Injection port temperature | 250° C. |
| Detection temperature | 250° C. |
| Split ratio | 50:1 |

### Comparative Example 1

The same operation as that of Example 1 was performed, except that a mixed liquid having an acetic acid concentration of 8.0% by mass and a water concentration of 92.0% by mass with respect to the total mass of the mixed liquid was used as the mixed liquid, and the concentration of impurities in the obtained crystals was measured. The obtained results are shown in Table 2.

### Comparative Example 2

The same operation as that of Example 1 was performed, except that a mixed liquid having an acetic acid concentration of 8.0% by mass, a water concentration of 91.95% by mass, and a methacrylic acid concentration of 0.05% by mass, with respect to the total mass of the mixed liquid was used as the mixed liquid, and the concentration of impurities in the obtained crystals was measured. The obtained results are shown in Table 2.

### Comparative Example 3

The same operation as that of Example 1 was performed, except that a mixed liquid having an acetic acid concentration of 8.0% by mass, a water concentration of 91.40% by mass, and a methacrylic acid concentration of 0.60% by mass, with respect to the total mass of the mixed liquid was used as the mixed liquid, and the concentration of impurities in the obtained crystals was measured. The obtained results are shown in Table 2.

### Comparative Example 4

The same operation as that of Example 1 was performed, except that a mixed liquid having an acetic acid concentration of 8.0% by mass, a water concentration of 91.20% by mass, and a methacrylic acid concentration of 0.80% by mass, with respect to the total mass of the mixed liquid was used as the mixed liquid, and the concentration of impurities in the obtained crystals was measured. The obtained results are shown in Table 2.

TABLE 2

| | | | | Concentrated effluent attached to | | |
|---|---|---|---|---|---|---|
| | Mixed liquid (composition) | | | crystals (composition) | | |
| | | | Methacrylic | | Methacrylic | Total |
| | Water | Acetic acid | acid | Acetic acid | acid | concentration |
| | (% by mass) | (% by mass) | (% by mass) | (ppm) | (ppm) | (ppm) |
| Example 1 | 91.8 | 8.0 | 0.2 | 28195 | 681 | 28876 |
| Comparative | 92.0 | 8.0 | 0.0 | 31649 | 0 | 31649 |
| Example 1 | | | | | | |
| Comparative | 91.95 | 8.0 | 0.05 | 36535 | 253 | 36788 |
| Example 2 | | | | | | |
| Comparative | 91.4 | 8.0 | 0.6 | 30648 | 2296 | 32944 |
| Example 3 | | | | | | |
| Comparative | 91.2 | 8.0 | 0.8 | 39922 | 3962 | 43884 |
| Example 4 | | | | | | |

From the results shown in Table 2, it is understood that when a separation operation of water-containing crystals is performed in Comparative Examples 1 to 4 by using mixed liquids including a specific amount of methacrylic acid as in Example 1, the concentration of acetic acid attached to the crystals can be reduced. Furthermore, when the total concentrations of acetic acid and methacrylic acid attached to the crystals are compared, it is also understood that the total concentration of acetic acid and methacrylic acid in Example 1 is lower than the total concentration of acetic acid and methacrylic acid in any of Comparative Examples 1 to 4.

### INDUSTRIAL APPLICABILITY

According to the present invention, a method for separating water-containing crystals, by which high-purity water can be separated from a mixed liquid including methacrylic acid, acetic acid, and water by a simpler method, and a method for producing methacrylic acid using this method for separating water-containing crystals, and a method for producing a methacrylic acid ester using the method for producing methacrylic acid, can be provided.

What is claimed is:

1. A method of separating water-containing crystals from a mixed liquid, comprising:
forming water-containing crystals from the mixed liquid, the mixed liquid comprising water, acetic acid, and methacrylic acid; and
separating the water-containing crystals from the mixed liquid,
wherein a weight ratio of methacrylic acid with respect a total weight of water, acetic acid and methacrylic acid in the mixed liquid is 0.09 wt % or more and 0.06 wt % or less.

2. The method according to claim 1, wherein a weight ratio of water with respect to the total weight of water, acetic acid and methacrylic acid in the mixed liquid is more than 79.94 wt % and 99.86 wt % or less.

3. The method according to claim 1, wherein a weight ratio of acetic acid with respect to the total weight of water, acetic acid and methacrylic acid in the mixed liquid is 0.05 wt % or more and 20.00 wt % or less.

4. The method according to claim 1, wherein the forming the water-containing crystals comprising cooling the mixed liquid to form the water-containing crystals.

5. The method according to claim 4, wherein a cooling temperature of the mixed liquid is −15° C. or higher and 10° C. or lower.

6. A method of producing methacrylic acid, comprising separating water by the method according to claim 1.

7. A method of producing a methacrylic acid ester, comprising:
producing methacrylic acid by the method according to claim 6; and
producing the methacrylic acid ester from the methacrylic acid and methanol.

8. The method according to claim 1, wherein a water content of the water-containing crystals is 80 wt % or more and 100 wt % or less.

9. The method according to claim 1, wherein a water content of the water-containing crystals is 90 wt % or more and 100 wt % or less.

10. The method according to claim 1, wherein a water content of the water-containing crystals is 95 wt % or more and 100 wt % or less.

11. The method according to claim 5, wherein, in the cooling the mixed liquid, a cooling rate of the mixed liquid is 0.01 K/min or more and 2.00 K/min or less.

12. The method according to claim 5, wherein, in the cooling the mixed liquid, a cooling rate of the mixed liquid is 0.02 K/min or more and 1.00 K/min or less.

13. The method according to claim 5, wherein, in the cooling the mixed liquid, a cooling rate of the mixed liquid is 0.04 K/min or more and 0.50 K/min or less.

14. The method according to claim 5, wherein a duration for maintaining the cooling temperature is 20 minutes or more and 5 hours or less.

15. The method according to claim 5, wherein a duration for maintaining the cooling temperature is 40 minutes or more and 4 hours or less.

16. The method according to claim 5, wherein a duration for maintaining the cooling temperature is 60 minutes or more and 3 hours or less.

17. The method according to claim 1, wherein, in the separating the water-containing crystals, the water-containing crystals are separated by filtering a slurry containing the water-containing crystals and a mother liquid containing acetic acid and methacrylic acid.

18. The method according to claim 1, wherein the water-containing crystals includes acetic acid in an amount of more than 0 ppm to 4000 ppm or less.

19. The method according to claim 1, wherein the water-containing crystals includes acetic acid in an amount of more than 0 ppm to 3000 ppm or less.
